(19) United States
(12) Patent Application Publication
(10) Pub. No.: US 2012/0225112 A1
(43) Pub. Date: Sep. 6, 2012

(54) Method and Apparatus for Alcohol Administration

(76) Inventor: Mark Stephen Saia, Rohnert Park, CA (US)

(21) Appl. No.: 13/412,355

(22) Filed: Mar. 5, 2012

Related U.S. Application Data
(60) Provisional application No. 61/448,731, filed on Mar. 3, 2011.

(51) Int. Cl. A61K 31/045; A61P 25/32; B32B 3/02; B32B 1/06; B32B 3/26; A61K 9/70; C09J 7/02

(52) U.S. Cl. 424/449; 514/724; 428/354; 428/35.2; 428/317.3; 428/68

(57) ABSTRACT

The method and apparatus for the administration of alcohol to a person provides an alcohol “patch” in the form of an adhesive patch or bandage-like structure bearing a quantity of alcohol for transdermal delivery to the user. Permeation enhancers, either chemical or structural, may be included for increased efficacy of delivery of the alcohol to the user. The alcohol to be delivered via the inventive apparatus may be plain ethyl alcohol (ethanol) of any suitable concentration or proof. Alternatively, the alcohol may be in the form of various distilled or undistilled spirits.

### CROSS REFERENCE TO RELATED APPLICATIONS

The present application claims the benefit of the filing date of U.S. Provisional Patent Application Ser. No. 61/448,731, filed Mar. 3, 2011. The foregoing application is incorporated by reference in its entirety as if fully set forth herein.

### STATEMENT REGARDING FEDERALLY SPONSORED RESEARCH OR DEVELOPMENT

Not applicable.

### REFERENCE TO A MICROFICHE APPENDIX

Not applicable.

### TECHNICAL FIELD

The present invention relates generally to alcoholic beverages and alcohol consumption, and more particularly to an improved method and apparatus for the administration of alcohol to a person.

### BACKGROUND INFORMATION AND DISCUSSION OF RELATED ART

U.S. Pat. No. 4,804,541 to Nichols discloses a method, composition, and article for use in transdermal or percutaneous administration to humans of systemically active medicaments in the form of a solution in benzyl alcohol.

U.S. Pat. No. 5,899,856 to Schoendorfer, et al. describes a dermal patch detecting long-term alcohol consumption and method of use. A non-occlusive dermal patch for collecting vapor phase perspiration from a subject's skin and retaining an analyte such as ethanol in the perspiration is disclosed. In addition, a method of collecting vapor phase perspiration containing an analyte such as ethanol over a period up to several days and detecting the analyte to determine the wearer's consumption of the analyte during the period when the patch was worn is disclosed.

U.S. Pat. No. 5,948,433 to Burton, et al. teaches transdermal patches, including a backing layer, a liner layer, and a monolithic adhesive and drug-containing layer between the backing layer and the liner layer. The drug-containing adhesive layer includes polyisobutylene, a plasticizer for the polyisobutylene in which the ratio of the plasticizer and the polyisobutylene is less than about 0.8 and at least 5% of a filler. The drug so utilized is moderately soluble in the plasticizer.

U.S. Pat. No. 6,328,992 to Brooke, et al. discloses a Cannabinoid patch and method for cannabis transdermal delivery. A transdermal structure is provided for delivering cannabis chemical(s) to one's bloodstream. The structure comprises a backing layer which carries the cannabis chemical(s). The chemicals are contained in a film on the backing layer or within a cavity formed in the backing layer. Alternatively, an opening in a secondary layer that overlies the backing layer may be used to create the cavity. The structure is applied to one's skin so that the cannabis chemicals are in contact with the skin A polymer material which is mixed with the cannabis and placed in the cavity or a membrane over the cavity may be used to control the flow of cannabis chemical (s) into the bloodstream. In an alternative embodiment, a porous material impregnated with cannabis chemical (s) may be used to hold the chemical(s) in the cavity. Because of the relatively slow transdermal flow rate of cannabis materials, it is preferred to utilize permeation enhancers in conjunction with the cannabis carrier or reservoir matrixes or skin contacting adhesive layers.

U.S. Pat. No. 6,974,588 to Miranda, et al. describes a transdermal patch for administering a volatile liquid drug, such as nicotine, transdermally to a patient comprising a four-layer laminated composite of: a top drug impermeable backing layer; a pressure sensitive silicone adhesive layer containing the drug; a pressure sensitive acrylic adhesive layer also containing the drug; and a removable siliconized release liner layer. Also disclosed is a method for treating a person for nicotine dependence and particularly for treating a woman for nicotine dependence.

U.S. Pat. No. 7,611,481 to Cleary, et al. teaches a dermal, transdermal, mucosal or transmucosal delivery device includes a backing layer overlying an ingredient containing reservoir, and having a microprotrusion array attached thereto, a cover for the reservoir having at least one opening therethrough, an adhesive layer and a liner layer. Upon removal of the liner layer, the device may be placed over the desired area of the skin or mucosa and adhesively applied thereto allowing the ingredients to flow from the reservoir through the at least one opening to the skin or mucosa.

The foregoing patents reflect the current state of the art of which the present inventor is aware. Reference to, and discussion of, these patents is intended to aid in discharging Applicant's acknowledged duty of candor in disclosing information that may be relevant to the examination of claims to the present invention. However, it is respectfully submitted that none of the above-indicated patents disclose, teach, suggest, show, or otherwise render obvious, either singly or when considered in combination, the invention described and claimed herein.

### SUMMARY OF THE INVENTION

The present invention provides an improved method and apparatus for the administration of alcohol to a person. Typically, a person who desires to achieve the effects of drinking an alcoholic beverage must physically consume an alcoholic beverage. Unfortunately, this is not always convenient or socially acceptable.

The method and apparatus of the present invention provides an alcohol “patch” in the form of an adhesive patch or bandage-like structure bearing a quantity of alcohol for transdermal delivery to the user. Permeation enhancers, either chemical or structural, may be included to facilitate the penetration of the alcohol through the skin, and for increased efficacy of delivery of the alcohol to the bloodstream of the user.

The alcohol to be delivered to the user via the inventive apparatus may be plain ethyl alcohol (ethanol) of any suitable concentration or proof. Alternatively, the alcohol may be in the form of various distilled or undistilled spirits (e.g., vodka, gin, rum, whiskey, tequila, and the like), again in suitable proof.

The transdermal patch is designed to deliver a regulated dose of alcohol through the skin and into the bloodstream of the user. The adhesive patch can be applied to the skin of the user on the arm, shoulder, leg, or other body part. Once through the skin, the alcohol easily penetrates the blood vessels and is distributed throughout the body. Once in the bloodstream, the person experiences the same euphoric effects as when an alcoholic beverage is consumed and the alcohol is ingested through the stomach. Generally, the effects of the alcohol begin within 30 to 45 minutes after the patch is applied, with the effects of a single patch lasting up to four hours or longer.

Use of the inventive transdermal patch produces the same effect as ingesting liquor. The inventive patch is convenient, can be carried anywhere, requires no refrigeration, and can be used discreetly and privately.

The transdermal patch may be used by those who don't necessarily enjoy the taste of liquor, but nonetheless desire the effects. Use of the patch may be healthier than ingesting liquor through the stomach, as the alcohol enters directly into the bloodstream, and avoids any ill effects to the user's stomach. In addition, when plain ethyl alcohol is used in the patch, the alcohol contains far fewer impurities than with the use of traditional liquors.

Many people that drink liquor will find a recreational use for the inventive transdermal patch. Furthermore, the patch may be used in the medical field as a delivery system useful for alcohol detoxification, as the quantity of alcohol can be regulated.

It is therefore an object of the present invention to provide a new and improved apparatus for the administration of alcohol to a person.

It is another object of the present invention to provide a new and improved apparatus for the delivery of plain ethyl alcohol (ethanol) of any suitable quantity, concentration or proof to a user.

A further object or feature of the present invention is a new and improved method to deliver alcohol to a person who desires to achieve the effects of drinking an alcoholic beverage without physically consuming an alcoholic beverage.

Other novel features which are characteristic of the invention, as to organization and method of operation, together with further objects and advantages thereof will be better understood from the following description considered in connection with the accompanying drawings, in which preferred embodiments of the invention are illustrated by way of example. It is to be expressly understood, however, that the drawings are for illustration and description only and are not intended as a definition of the limits of the invention. The various features of novelty which characterize the invention are pointed out with particularity in the claims annexed to and forming part of this disclosure. The invention resides not in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is neither intended to define the invention of this application, which is measured by the claims, nor is it intended to be limiting as to the scope of the invention in any way.

Certain terminology and derivations thereof may be used in the following description for convenience in reference only, and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

### BRIEF DESCRIPTION OF THE DRAWINGS

The invention will be better understood and objects other than those set forth above will become apparent when consideration is given to the following detailed description thereof. Such description makes reference to the annexed drawing wherein:

FIG. 1 is a side elevation cross-sectional view of a transdermal patch for alcohol administration of this invention.

### DETAILED DESCRIPTION OF THE INVENTION

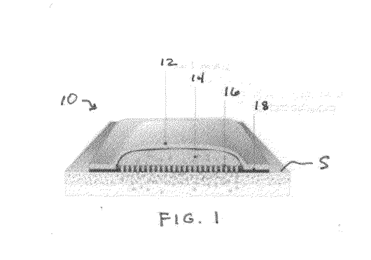

FIG. 1 illustrates a first preferred embodiment of a transdermal bandage or patch 10 for the administration of alcohol to a user, preferably including a backing layer 12, alcohol reservoir 14, release membrane 16, and contact adhesive layer 18. Backing layer 12 is preferably constructed of a flexible plastic, polymer, or other suitable material that is impermeable to alcohol, completely covers the alcohol reservoir 14, and may be clear, translucent, opaque or colored. Alcohol reservoir 14 may be a simple void or cavity between the backing layer 12 and release membrane 16, or may include a pad of preferably nonwoven material such as nylon, rayon, cotton, or other material that is absorbent and will contain the liquid alcohol prior to use and yet permit delivery of the alcohol when the patch 10 is applied to the skin of the user. The quantity of alcohol contained in the reservoir 14 for delivery to the user can of course vary, but may be in a dose of two grams or more. Release membrane 16 may be constructed of nylon, polymer, thermoplastic fluoropolymer such as pvdf, nitro cellulose, or other suitable material that prevents the free flow and loss of the alcohol prior to the application of the patch 10 to the user's skin, but permits passage and delivery of the alcohol from the reservoir 14 to and through the exposed skin S of the user when applied. Contact adhesive layer 18 surrounds the release membrane 16 and forms a seal with the skin S to prevent leakage of alcohol from around the periphery of release membrane 16. The contact adhesive may be a pressure sensitive water based or solvent based acrylic or silicone adhesive, or other suitable adhesive that will at least temporarily hold the patch structure in place on the user's skin when applied, and yet be selectively releasable from the skin when desired, as when the quantity of alcohol from the patch has been completely delivered to the user through the skin. The entire transdermal patch structure 10 is preferably packaged in an easily opened sealed envelope or other package for the convenience of the user.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Therefore, the above description and illustrations should not be construed as limiting the scope of the invention, which is defined by the appended claims.

What is claimed as invention is:

1. An apparatus for the administration of alcohol to a person, said apparatus comprising:
an adhesive patch structure bearing a quantity of alcohol for transdermal delivery to a user.

2. The apparatus for the administration of alcohol to a person of claim 1 wherein said alcohol comprises ethyl alcohol.

3. The apparatus for the administration of alcohol to a person of claim 1 wherein said alcohol comprises distilled spirits.

4. The apparatus for the administration of alcohol to a person of claim 1 wherein said adhesive patch includes permeation enhancers.

5. The apparatus for the administration of alcohol to a person of claim 1 wherein said adhesive patch comprises a backing layer, alcohol reservoir, release membrane, and contact adhesive layer.

6. The apparatus for the administration of alcohol to a person of claim 5 wherein said backing layer comprises a flexible material that is impermeable to alcohol.

7. The apparatus for the administration of alcohol to a person of claim 5 wherein said backing layer completely covers said alcohol reservoir.

8. The apparatus for the administration of alcohol to a person of claim 5 wherein said alcohol reservoir comprises a cavity between said backing layer and said release membrane.

9. The apparatus for the administration of alcohol to a person of claim 5 wherein said alcohol reservoir comprises a pad of material that is absorbent and will contain said alcohol prior to use and permit delivery of said alcohol when said patch is applied to the skin of a user.

10. The apparatus for the administration of alcohol to a person of claim 5 wherein said alcohol reservoir comprises a dose of at least two grams of alcohol.

11. The apparatus for the administration of alcohol to a person of claim 5 wherein said release membrane is constructed of a material that prevents the free flow and loss of said alcohol prior to the application of said patch to a user's skin, but permits passage and delivery of said alcohol from said alcohol reservoir to and through the skin of user when applied.

12. The apparatus for the administration of alcohol to a person of claim 5 wherein said contact adhesive layer surrounds said release membrane and forms a seal with the skin of a user to prevent leakage of alcohol from around the periphery of said release membrane.

13. The apparatus for the administration of alcohol to a person of claim 5 wherein said contact adhesive layer comprises an adhesive that will at least temporarily hold said patch structure in place on a user's skin when applied.

14. The apparatus for the administration of alcohol to a person of claim 5 wherein said contact adhesive layer is selectively releasable from the skin of a user when said alcohol from said patch has been completely delivered to the user.
